(19) United States
(12) Patent Application Publication
(10) Pub. No.: US 2023/0240574 A1
(43) Pub. Date: Aug. 3, 2023

(54) Biofeedback System

(71) Applicants: Simon Salem, Moorpark, CA (US); Vasim Hanna-Salem, Moorpark, CA (US)

(72) Inventors: Simon Salem, Moorpark, CA (US); Vasim Hanna-Salem, Moorpark, CA (US)

(21) Appl. No.: 17/591,737

(22) Filed: Feb. 3, 2022

(51) Int. Cl. A61B 5/16; A61B 5/00; A61B 5/024; A61B 5/1171; G06V 40/16

(52) U.S. Cl. CPC A61B 5/165; A61B 5/746; A61B 5/4803; A61B 5/02438; A61B 5/743; A61B 5/7435; A61B 5/7405; A61B 5/1176; A61B 5/0013; A61B 5/0077; G06V 40/176; A61B 2562/0204

(57) ABSTRACT

A biofeedback system includes a personal electronic device storing operational software including a physiological feedback program. A plurality of biometric sensors is provided and each of the biometric sensors is in remote communication with the personal electronic device. Each of the biometric sensors is strategically located such that each of the biometric sensors is facilitated to sense a respective one of the user's heart rate, the user's vocal volume, the user's vocal pitch and the user's facial expressions. The personal electronic device emits an alert when the physiological feedback program determines that the data received from any of the biometric sensors exceeds a pre-determined threshold to facilitate the user to monitor their physical state and emotional state.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to biofeedback devices and more particularly pertains to a new biofeedback device for monitoring a user's physical and emotional state. The device includes a personal electronic device which stores a physiological feedback program and a plurality of biometric sensors which are strategically oriented on and around the user. The personal electronic device receives data from each of the biometric sensors to and the physiological feedback program emits an alert when any of the user's physiological responses exceeds a pre-determined threshold.

### (2) Description of Related Art Including Information Disclosed Under 37 CFR 1.97 and 1.98

The prior art relates to biofeedback devices including a variety of physiological monitoring devices that include wearable sensors that monitor various physiological responses of a user which transit data to a remote processing unit for subsequent analysis. The prior art discloses a biofeedback device that includes a microphone for recording a user's voice and analysis software for analyzing the user's voice. In no instance does the prior art disclose a biofeedback device that includes a personal electronic device in communication with a plurality of biometric sensors and which includes vocal analysis.

### BRIEF SUMMARY OF THE INVENTION

An embodiment of the disclosure meets the needs presented above by generally comprising a personal electronic device storing operational software including a physiological feedback program. A plurality of biometric sensors is provided and each of the biometric sensors is in remote communication with the personal electronic device. Each of the biometric sensors is strategically located such that each of the biometric sensors is facilitated to sense a respective one of the user's heart rate, the user's vocal volume, the user's vocal pitch and the user's facial expressions. The personal electronic device emits an alert when the physiological feedback program determines that the data received from any of the biometric sensors exceeds a pre-determined threshold to facilitate the user to monitor their physical state and emotional state.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a biofeedback system according to an embodiment of the disclosure.

FIG. 2 is a logic tree view of an embodiment of the disclosure.

FIG. 3 is a front view of personal electronic device of an embodiment of the disclosure.

FIG. 4 is a schematic view of an embodiment of the disclosure.

### DETAILED DESCRIPTION OF THE INVENTION

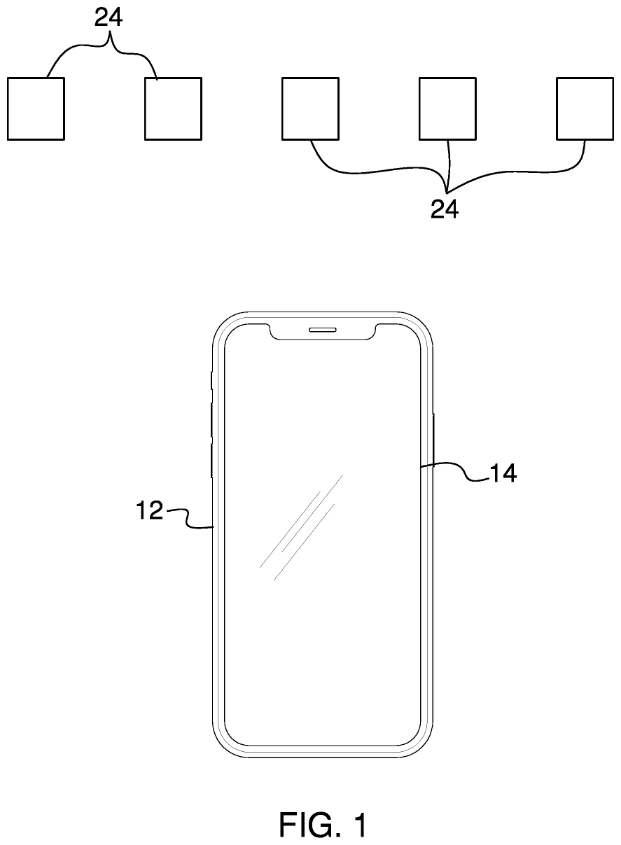

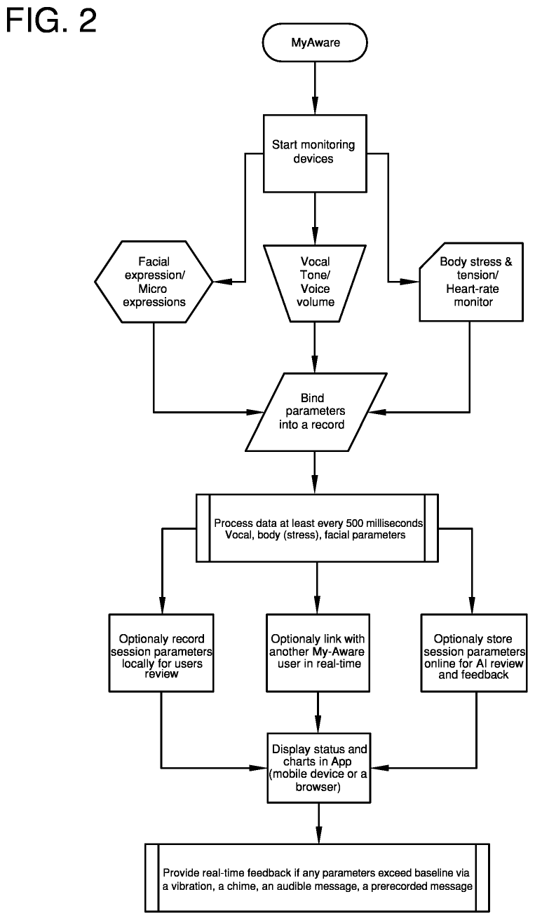

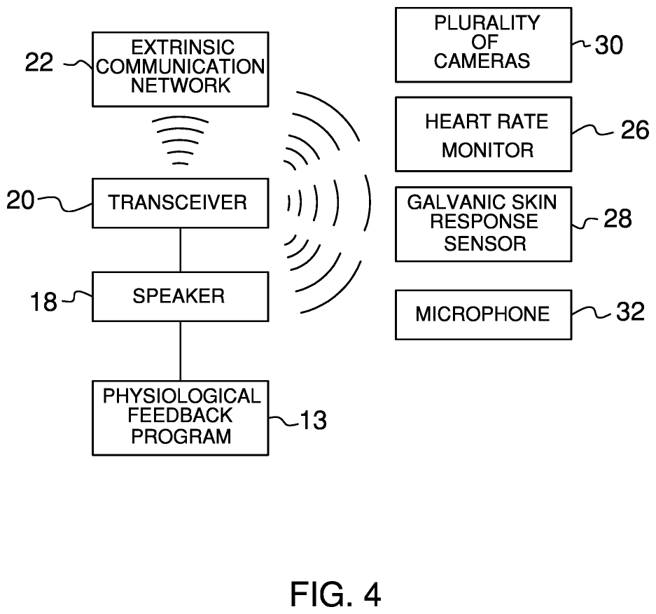

With reference now to the drawings, and in particular to FIGS. 1 through 4 thereof, a new biofeedback device embodying the principles and concepts of an embodiment of the disclosure and generally designated by the reference numeral 10 will be described.

As best illustrated in FIGS. 1 through 4, the biofeedback system 10 generally comprises a personal electronic device 12 storing operational software including a physiological feedback program 13. The personal electronic device 12 includes a display 14 for displaying indicia 16 comprising icons, text and images related to the physiological feedback program 13. In this way the display 14 facilitates a user to observe data related to the physiological feedback program 13. The personal electronic device 12 includes a speaker 18 and the speaker 18 is actuated to emit an audible alert when the physiological feedback program 13 records physiological responses that exceed a pre-determined threshold which was established by the user. In this way the speaker 18 can audibly alert the user to the physiological response that exceeds the pre-determined threshold.

The operational software includes a facial recognition program 13 and the personal electronic device 12 includes a transceiver 20. The personal electronic device 12 may comprise a smart phone or other similar type of device 12. The display 14 may comprise a liquid crystal display or other type of electronic display and the transceiver 20 may comprise a radio frequency transceiver. Furthermore, the transceiver 20 may be in communication with an extrinsic communication network 22, such as the internet or a cellular phone network, and the transceiver 20 may employ Bluetooth communication protocols. The user enters a heart rate baseline, a galvanic skin response baseline, a vocal pitch baseline and a vocal volume baseline into the physiological feedback program 13.

A plurality of biometric sensors 24 is provided and each of the biometric sensors 24 is in remote communication with the personal electronic device 12. Each of the biometric sensors 24 is strategically located such that each of the biometric sensors 24 is facilitated to sense a respective one of the user's heart rate, galvanic skin response, the user's vocal volume, the user's vocal pitch and the user's facial expressions. The physiological feedback program 13 analyzes data received from each of the biometric sensors 24 and the personal electronic device 12 emits an alert when the physiological feedback program 13 determines that the data received from any of the biometric sensors 24 exceeds a pre-determined threshold. In this way the biometric sensors 24 facilitate the user to monitor their physical state and emotional state.

The plurality of biometric sensors 24 includes a heart rate monitor 26 which wearable on the user to detect the user's heart rate. The heart rate monitor 26 is in wireless communication with the transceiver 20 in the personal electronic device 12 thereby facilitating the physiological feedback program 13 to record the user's heart rate. Additionally, the heart rate monitor 26 includes a galvanic skin response sensor 28 for sensing the user's skin conductance. The speaker 18 is actuated when the physiological feedback program 13 records a heart rate that exceeds a threshold heart rate determined by the user to alert the user that their heart rate has exceeded the threshold heart rate. Additionally, the speaker 18 is actuated when the physiological feedback program 13 records a galvanic skin response that exceeds a threshold galvanic skin response determined by the user to alert the user that their stress level is too high. The heart rate monitor 26 may comprise a smart watch of any conventional design or other similar type of electronic device that can be worn on the user's body.

The plurality of biometric sensors 24 includes a plurality of cameras 30 that is each positioned at strategic locations to capture imagery of the user's face. Each of the cameras 30 is in wireless communication with the transceiver 20 thereby facilitating the facial recognition software to analyze the imagery of the user's face. Additionally, the speaker 18 in the personal electronic device 12 emits the audible alert when the facial recognition software identifies facial expressions that correspond with pre-determined emotional states. In this way the speaker 18 alerts the user that the user is becoming emotionally agitated. The plurality of cameras 30 may be worn on the user's body or the plurality of cameras 30 may be integrated into the personal electronic device 12.

The plurality of biometric sensors 24 includes a microphone 32 that is strategically positioned to capture the user's voice. The microphone 32 is in wireless communication with the transceiver 20 and the microphone 32 may be integral component of the personal electronic device 12. The physiological feedback program 13 analyzes the user's voice recorded by the microphone 32. Furthermore, the speaker 18 in the personal electronic device 12 emits the audible alert when the physiological feedback program 13 determines that the volume of the user's voice exceeds a pre-determined threshold. In this way the speaker 18 alerts the user that the user is speaking too loudly. The speaker 18 in the personal electronic device 12 emits the audible alert when the physiological feedback program 13 determines that a pitch of the user's voice exceeds a pre-determined threshold. In this way the speaker 18 can alert the user that the user is becoming verbally excited.

In use, the user manually enters the heart rate baseline, the galvanic skin response baseline, the vocal pitch baseline and the vocal volume baseline into the physiological feedback program 13. Additionally, baseline facial expressions are recorded with various self portraits depicting various emotions the user might experience. The user subsequently employs the physiological feedback program 13 to determine which baseline facial expressions the user wishes to monitor. The baseline parameters are bound into a record and the physiological feedback program 13 records data from the biometric sensors 24 every 500.0 milliseconds into a database stored in the personal electronic device 12. In this way the user can monitor their biological responses to various different situations, thereby facilitating the user to develop coping skills and healthy behavior patterns. Furthermore, the database can be stored in a remote data storage device 12, such as a cloud server or other online data storage, for subsequent analysis, or the database can be shared with another personal electronic device 12.

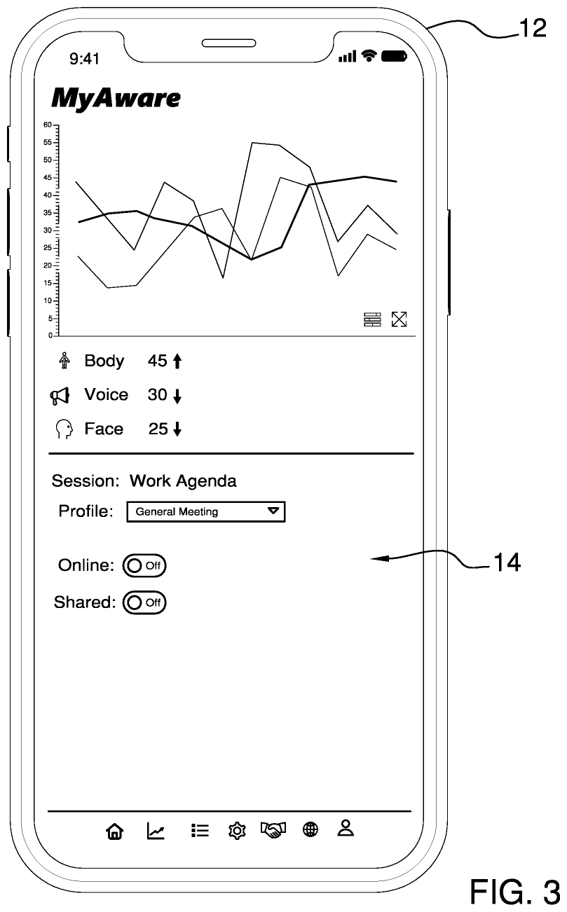

As is most clearly shown in FIG. 3, the physiological feedback program 13 compiles the data into various charts that are displayed on the display 14 for the user to interpret. The personal electronic device 12 may provide haptic feedback, such as vibration, in lieu of the speaker 18 emitting the audible alert. Additionally, the audible alert may include a chime tone or a pre-recorded verbal message. The user can create multiple baseline profiles in the physiological feedback program 13 to correspond to various situations the user might encounter. Furthermore, the user can copy baseline profiles, modify the baseline profiles, and perform various types of data analysis of the baseline profiles. Each session of biometric analysis can be recorded locally for the user to review, recorded online for third party analysis or shared in real time with a third party's electronic device 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A biofeedback system for alerting a user when various physiological responses have risen beyond a pre-determined threshold, said system comprising:
a personal electronic device storing operational software including a physiological feedback program; and
a plurality of biometric sensors, each of said biometric sensors being in remote communication with said personal electronic device, each of said biometric sensors being strategically located such that each of said biometric sensors is facilitated to sense a respective one of the user's heart rate, the user's vocal volume, the user's vocal pitch and the user's facial expressions, said physiological feedback program analyzing data received from each of said biometric sensors, said personal electronic device emitting an alert when said physiological feedback program determines that said data received from any of said biometric sensors exceeds a pre-determined threshold wherein said biometric sensors are configured to facilitate the user to monitor their physical state and emotional state.

2. The assembly according to claim 1, wherein:
said personal electronic device includes a display for displaying indicia comprising icons, text and images related to said physiological feedback program wherein said display is configured to facilitate a user to observe data related to said physiological feedback program;
said personal electronic device includes a speaker, said speaker being actuated to emit an audible alert when said physiological feedback program records physiological responses that exceeds a pre-determined threshold which was established by the user wherein said speaker is configured to audibly alert the user to the physiological response that exceeds the pre-determined threshold;
said operational software includes a facial recognition program; and
said personal electronic device includes a transceiver.

3. The assembly according to claim 2, wherein said plurality of biometric sensors includes a heart rate monitor wearable on the user wherein said heart rate monitor is configured to detect the user's heart rate, said heart rate monitor being in wireless communication with said transceiver in said personal electronic device thereby facilitating said physiological feedback program to record the user's heart rate.

4. The assembly according to claim 3, wherein said speaker is actuated when said physiological feedback program records a heart rate that exceeds a threshold heart rate determined by the user wherein said heart rate monitor is configured to alert the user that their heart rate has exceeded the threshold heart rate.

5. The assembly according to claim 2, wherein said plurality of biometric sensors includes a plurality of cameras, each of said cameras being positioned at strategic locations wherein each of said cameras is configured to capture imagery of the user's face, each of said cameras being in wireless communication with said transceiver thereby facilitating said facial recognition software to analyze the imagery of the user's face.

6. The assembly according to claim 5, wherein said speaker in said personal electronic device emitting said audible alert when said facial recognition software identifies facial expressions that correspond with pre-determined emotional states wherein said speaker is configured to alert the user that the user is becoming emotionally agitated.

7. The assembly according to claim 2, wherein said plurality of biometric sensors includes a microphone being strategically positioned wherein said microphone is configured to capture the user's voice, said microphone being in wireless communication with said transceiver, said physiological feedback program analyzing the user's voice recorded by said microphone.

8. The assembly according to claim 7, wherein said speaker in said personal electronic device emits said audible alert when said physiological feedback program determines that the volume of the user's voice exceeds a pre-determined threshold wherein said speaker is configured to alert the user that the user is speaking too loudly.

9. The assembly according to claim 7, wherein said speaker in said personal electronic device emits said audible alert when said physiological feedback program determines that a pitch of the user's voice exceeds a pre-determined threshold wherein said speaker is configured to alert the user that the user is becoming verbally excited.

10. A biofeedback system for alerting a user when various physiological responses have risen beyond a pre-determined threshold, said system comprising:
a personal electronic device storing operational software including a physiological feedback program, said personal electronic device including a display for displaying indicia comprising icons, text and images related to said physiological feedback program wherein said display is configured to facilitate a user to observe data related to said physiological feedback program, said personal electronic device including a speaker, said speaker being actuated to emit an audible alert when said physiological feedback program records physiological responses that exceeds a pre-determined threshold which was established by the user wherein said speaker is configured to audibly alert the user to the physiological response that exceeds the pre-determined threshold, said operational software including a facial recognition program, said personal electronic device including a transceiver; and
a plurality of biometric sensors, each of said biometric sensors being in remote communication with said personal electronic device, each of said biometric sensors being strategically located such that each of said biometric sensors is facilitated to sense a respective one of the user's heart rate, the user's vocal volume, the user's vocal pitch and the user's facial expressions, said physiological feedback program analyzing data received from each of said biometric sensors, said personal electronic device emitting an alert when said physiological feedback program determines that said data received from any of said biometric sensors exceeds a pre-determined threshold wherein said biometric sensors are configured to facilitate the user to monitor their physical state and emotional state, said plurality of biometric sensors including:
a heart rate monitor wearable on the user wherein said heart rate monitor is configured to detect the user's heart rate, said heart rate monitor being in wireless communication with said transceiver in said personal electronic device thereby facilitating said physiological feedback program to record the user's heart rate, said speaker being actuated when said physiological feedback program records a heart rate that exceeds a threshold heart rate determined by the user wherein said heart rate monitor is configured to alert the user that their heart rate has exceeded the threshold heart rate; and
a plurality of cameras, each of said cameras being positioned at strategic locations wherein each of said cameras is configured to capture imagery of the user's face, each of said cameras being in wireless communication with said transceiver thereby facilitating said facial recognition software to analyze the imagery of the user's face, said speaker in said personal electronic device emitting said audible alert when said facial recognition software identifies facial expressions that correspond with pre-determined emotional states wherein said speaker is configured to alert the user that the user is becoming emotionally agitated; and
a microphone being strategically positioned wherein said microphone is configured to capture the user's voice, said microphone being in wireless communication with said transceiver, said physiological feedback program analyzing the user's voice recorded by said microphone, said speaker in said personal electronic device emitting said audible alert when said physiological feedback program determines that the volume of the user's voice exceeds a pre-determined threshold wherein said speaker is configured to alert the user that the user is speaking too loudly, said speaker in said personal electronic device emitting said audible alert when said physiological feedback program determines that a pitch of the user's voice exceeds a pre-determined threshold wherein said speaker is configured to alert the user that the user is becoming verbally excited.
